(19) United States
(12) Patent Application Publication
(10) Pub. No.: US 2025/0041287 A1
(43) Pub. Date: Feb. 6, 2025

(54) NICOTINE CHEWING GUM

(71) Applicant: MCNEIL AB, Helsingborg (SE)

(72) Inventors: Martin EDMAN, Klippan (SE); Jill NILGARD, Helsingborg (SE)

(21) Appl. No.: 18/701,457

(22) PCT Filed: Oct. 20, 2022
(86) PCT No.: PCT/EP2022/079258

§ 371 Date: Apr. 15, 2024

(30) Foreign Application Priority Data
Oct. 21, 2021 (SE) 2151282-7

(51) Int. Cl. A61K 31/465; A23G 4/06; A23G 4/10; A23G 4/12; A23G 4/20; A61K 9/68; A61K 47/02; A61K 47/26; A61K 47/58

(52) U.S. Cl. CPC A61K 31/465; A23G 4/064; A23G 4/10; A23G 4/126; A23G 4/20; A61K 9/0058; A61K 47/02; A61K 47/26; A61K 47/585

(57) ABSTRACT

The invention relates to a nicotine chewing gum giving an immediate release and uptake of nicotine, as well as an extended release and uptake of nicotine. The invention further describes suitable manufacturing processes for such chewing gum formulations, as well as the use of the chewing gum for the treatment of a human being suffering from cravings from tobacco and/or e-cigarette dependency.

### FIELD OF INVENTION

This invention relates to a nicotine chewing gum giving an immediate release and uptake of nicotine and an extended release and uptake of nicotine, as well as suitable manufacturing processes for such chewing gum formulations. The invention further relates to the use of such chewing gums for the treatment of a human being suffering from cravings from tobacco and/or e-cigarette dependency.

### BACKGROUND OF INVENTION

According to WHO about six million people die from smoking related diseases each year, even though there are products (medicines) on the market to help a smoker to quit smoking; products such as e.g., nicotine comprising chewing gums, lozenges, sprays and transdermal patches.

A traditional way to produce a nicotine chewing gum is to create a complex of nicotine with a cation exchange resin and to add this complex to a chewing gum formulation.

Such chewing gums have been available on the market for many years, sold under, for example the trademark Nicorette®. However, there are consumers that are looking for nicotine products (medicines) that could provide faster craving relief, closer to the craving relief of a cigarette and, thus, there is still an opportunity to develop new nicotine chewing gums that could satisfy this population using nicotine in any form, including tobacco and e-cigarettes.

One product on the market giving rise to a faster craving relief is the Nicorette™ QuickMist™, which is a mouth spray to be applied to the oral mucosa from which the nicotine compound is readily absorbed into the blood stream to give a fast-craving relief. In the Nicorette™ QuickMist™ product, nicotine is in the free form and due to the buffering capacity of the system nicotine is rapidly passing the mucosa to give a rapid craving relief.

Thus, it would be obvious for a person skilled in the art to make a hybrid between the liquid QuickMist™ spray and a chewing gum. However, such a product would provide a number of problems including stability of the nicotine, i.e., nicotine is not stable as a liquid, manufacturing and storage problems. If you try to prepare a chewing gum with a liquid inside, the chewing gum will slowly be dissolved and the components present within the liquid and chewing gum will mix causing unsuitable chemical reactions.

A chewing gum comprising nicotine needs to be chewed upon and mixed with saliva to release nicotine through the mouth mucosa. Sprays like QuickMist™ use a liquid comprising nicotine as well as one or more buffers to increase the speed of transmucosal uptake.

It would be obvious for a person skilled in the art to propose a chewing gum with a liquid core. However, such a product is disadvantaged by a number of problems including the stability of the free form of nicotine normally used in QuickMist™.

Thus, there is a need to find an alternative nicotine replacement product with the same convenience as chewing gum and having the same fast uptake of nicotine that the QuickMist™ provides, while also preserving the mouthfeel to be pleasant.

WO2007/133141 discloses a nicotine chewing gum having a solid core comprising nicotine in any form, and at least one coating layer buffered with trometamol. WO2007/133141 discloses a chewing gum core that comprises a chewing gum base, however it is silent about liquid core.

WO2020/012009 discloses a nicotine chewing gum having a dot on the chewing gum, where the dot should give an immediate release of nicotine and a fast craving relief and the chewing gum an extended release. The problem with that invention being that it was impossible to manufacture the product, as well as it was very energy demanding. The ingredients present in the dot need to be in a liquid phase upon being placed onto the chewing gum, which needs energy. Additionally, this approach is dangerous for the people in the production plant since nicotine in a liquid is toxic, and nicotine and buffer cannot be present together. The present approach presents the problem that the nicotine will revert to its free form before the dot has been solidified.

The problem to be solved by this invention is to provide a product that gives a pleasant and acceptable mouth feeling, that provides a fast uptake of nicotine as well as an extended uptake of nicotine to aid a person to stop smoking. By providing such a product it is for the first time possible to provide a product which gives both an immediate and extended craving relief, as well as to provide a product that is possible to manufacture and store without stability problems.

### SUMMARY OF THE INVENTION

The inventors have been exploring the possibility to create a chewing gum giving rise to an immediate as well as an extended release and uptake of nicotine, which still has good taste and palatability, including being able to be manufactured. To provide an immediate release and uptake from a nicotine gum product, the inventors approach has been to apply nicotine in a more readily available nicotine salt form, such as nicotine bitartrate or nicotine ditartrate dihydrate comprised in a core present as a powder within a chewing gum. However, to have a core with a powder also means that the consumer upon chewing the gum will be exposed to a powder which normally is not a pleasant feeling. However, the invented powder has been designed to be dissolved in a fast immediate manner and, thus, give a pleasant taste and feeling.

The pKa for nicotine is approximately 7.8. It is well known that the uncharged nicotine (free base form) could more easily and faster enter biological membranes, such as the oral mucosa, compared to nicotine salt or nicotine in its positively charged acid form. Easier and faster permeation through biological membranes leads to a faster systemic uptake.

The average pH of the human saliva is normally just about 6-7.5.

By selecting and adding certain ingredients into the chewing gum powder core, a fast immediate uptake will occur into the mucosa without any unsuitable feelings of powders in the mouth. This would result in that approximately about 90% of the nicotine in the powder core would be converted to its' free base form after disintegration and dissolution of the powder in the saliva, to be readily absorbed into the systemic circulation to provide for a faster craving relief. The powder will be dissolved when the consumer is chewing on the gum within up to about 30 seconds and adsorbed into the mucosa to provide a fast craving relief, such as 15-30, 10 or 20 seconds. How fast the powder will be dissolved is dependent on how intensely the consumer is chewing on the gum.

Nicotine free base form is not suitable to directly be formulated into semisolid or solid dosage forms since the nicotine free base form is in a highly volatile liquid form at normal conditions. To circumvent that problem, a powder is preferable, for example, where nicotine is in its salt form. By utilizing a salt it is easier to control as well as easier when manufacturing the product.

To be able to provide a transient and suitable pH increase, there is a benefit of using a buffering system, such as one or more different buffer(s) (buffer species). Normally, buffering systems do not taste well and thus they need to be selected carefully and taste masked by e.g., sweeteners and flavors. In addition, the buffering capacity of one buffer (buffer species) might not be sufficient.

To provide an extended release of nicotine from a nicotine chewing gum coat, the use of nicotine bound to a resin such as nicotine polacrilex is suitable. Nicotine polacrilex is normally used in the medicated chewing gums available on the market. Such a medicated chewing gum would provide an extended release over a time up to about 20 to 45 minutes, depending on the usage. However, the release and uptake from these gums during the first 5-10 minutes of usage is somewhat limited, which now is solved by using a powder core with nicotine salt, which would give a fast uptake.

The invented nicotine chewing gum provides new features in one and the same product, compared to other currently available commercialized nicotine chewing gum products available on the market.

In a first aspect the invention relates to a nicotine chewing gum comprising:
i. a powder core comprising at least nicotine bitartrate and/or nicotine ditartrate dihydrate, sweetener(s), buffer(s) and polyol based filling agent(s), and
ii. a chewing gum coat comprising at least nicotine polacrilex, wherein the nicotine from the powder core is immediately released and the nicotine from the chewing gum coat is extendedly released.

The invention also relates to a nicotine chewing gum comprising:
i. a powder core comprising at least nicotine bitartrate and/or nicotine ditartrate dihydrate, sweetener(s), buffer(s) and polyol based filling agent(s), and
ii. a chewing gum coat comprising at least a gum base and nicotine polacrilex, wherein the nicotine bitartrate and/or nicotine ditartrate dihydrate from the powder core has an immediate release and the nicotine polacrilex from the gum coat has an extended release.

Optionally at least one polyol-based hard coating covers the chewing gum, comprising at least one sugar alcohol or mixtures of sugar alcohols,

### DETAILED DESCRIPTION OF EMBODIMENTS OF THE INVENTION

### Definitions

In the context of the present application and invention the following definitions apply:

The term “nicotine” refers to the amount (mg) of nicotine in any salt form or bound to any carrier calculated as the amount of corresponding free base per piece of gum.

The term “buffer(s)” refers to one or two different kinds of buffer species also differentiating the corresponding acid-base pair of a buffer system.

As used herein, the term “extended release” (“ER”) refers to formulations which are characterized by that the nicotine present in the chewing gum core will be released over an extended period of mastication normally for 20-45 minutes, the time a consumer or patient is chewing on (using) the gum. The release profile may be assessed via in vitro dissolution using techniques known to those skilled in the art (European Pharmacopeia 9.0, General chapters, method of analysis, 2.9.25 Dissolution for medication chewing gums, apparatus B).

The term “immediate release” (“IR”) as used herein is intended to mean the release of the nicotine comprised in the powder core of the gum to be available for fast oromucosal absorption. The rate of release of nicotine is not prolonged by means of a controlled release matrix or other such means but it is dependent of the disintegration and dissolution of the chewing gum core and water solubility of the nicotine salt upon chewing. As described herein, an “immediate release” means that the nicotine release starts within about 30 seconds after starting chewing on the gum, which is dependent on how intensely the consumer is chewing on the gum and might be shorter than 30 seconds as well.

The term “powder” is intended to mean a powder containing fine dry particles that do not aggregate and that stays in a stable powder form and having a mean particle size of less than 310 μm, calculated on the largest excipient present within the powder.

The term “chewing gum coat” is intended to mean a chewing gum coat that covers the complete powder core and protects the powder from being exposed to humidity; and by doing that the powder core stays stable before the chewing gum is chewed on. Upon chewing the gum, the powder core is exposed to saliva and dissolves rapidly as defined above and gives rise to a rapid fast craving relief.

The term “mouth feel” refers to the physical sensations in the mouth caused by food or drink, in this case chewing on a gum as distinct from taste. It is a fundamental sensory attribute which, along with taste and smell, determines the overall flavor and texture of the chewing gum. It is used to describe the perception on the palate, through chewing.

The calculation of the amount of nicotine present in the chewing gum core is calculated and most often expressed as the amount of the corresponding free base form of nicotine.

### The Chewing Gum

In one embodiment, the invention relates to a nicotine chewing gum comprising a powder core comprising at least nicotine bitartrate and/or nicotine ditartrate dihydrate, sweetener(s), buffer(s) and polyol based filling agent(s), and a gum coat comprising at least nicotine polacrilex, where the nicotine bitartrate and/or nicotine ditartrate dihydrate from the powder core is immediately released and the nicotine polacrilex from the chewing gum coat is extendedly released. The gum coat encapsulates the powder into the core and protects the powder from humidity until chewing occurs on the chewing gum. When chewed, the powder is released and rapidly dissolved, and a fast craving relief occurs.

The invention also relates to a nicotine chewing gum comprising a powder core comprising at least nicotine bitartrate and/or nicotine ditartrate dihydrate, sweetener(s), buffer(s) and polyol based filling agent(s), and a gum coat comprising at least a gum base and nicotine polacrilex, where the nicotine bitartrate and/or nicotine ditartrate dihydrate from the powder core has an immediate release and the nicotine polacrilex from the chewing gum coat has an extended release.

The nicotine chewing gum may further comprise at least one polyol-based hard coating that coats an exterior of the gum coat.

Optionally a polyol-based hard coating covers the chewing gum, comprising at least one sugar alcohol or mixtures of sugar alcohols.

It is well known for a person skilled in the art how to produce a traditional chewing gum.

One example is the process disclosed in the U.S. Pat. No. 3,877,468. Coated chewing gums have been disclosed in WO2006/124366 wherein polyol-based hard coatings in combination with an inner polymer coating comprising one or more sweeteners and/or flavors were described to improve the taste and the sensory acceptance of the chewing gum.

The powder core comprises nicotine. The nicotine present in the chewing gum powder core (calculated as the free base) may be from about 0.5 to about 5.0 mg, about 2.0 to about 4.0 mg, such as 0.5, 1.0, 1.25, 1.5, 1.75, 2.0, 2.25, 2.5, 2.75, 3.0, 3.25, 3.5, 3.75, 4.0, 4.25, 4.5 or 5.0 calculated per piece of gum. The nicotine will be released from the powder core when the user is chewing on the gum and the release will occur immediately, within, for example, about 30 seconds, such as 10 or 20 seconds.

The buffer(s) suitable to be use are selected from the group consisting of sodium carbonate, sodium bicarbonate, potassium carbonate, potassium bicarbonate, trometamol base (Tris base), or the corresponding conjugated acid of trometamol such as Trometamol hydrochloride (Tris HCl), trisodium phosphate, disodium hydrogenphosphate, sodium dihydrogen phosphate, tripotassium phosphate, dipotassium hydrogenphosphate, potassium dihydrogen phosphate and mixtures thereof. According to an embodiment, the buffer may be selected from the group consisting of sodium carbonate, sodium bicarbonate, trometamol base (Tris base) or the corresponding conjugated acid of trometamol such as Trometamol hydrochloride (Tris HCl), or mixtures thereof. In some specific embodiments sodium carbonate and/or sodium bicarbonate are used.

The nicotine in the core needs to be preserved from the atmosphere and by doing that no humidity will come in contact with the dry powder and thus the nicotine will be dry and stable.

The total amount of the powder core may be up to 80 mg, such as from 10-80 mg, such as 10, 15, 20, 25, 30, 35, 40, 45, 50, 55, 60, 65, 70, 75 or 80 mg, such as 20 mg to 60 mg. The limitation is when the dissolution of the powder will be too slow and the palatability less pleasant. The particle size of the powder is dependent on the ingredients. If the amount of the powder in the core is 80 mg then the chewing gum coat need to be about 950 mg to be able to load such a high amount of powder. However, if the powder in the core is 40 mg then 475 mg of the chewing gum coat should be enough.

The buffer(s) in the powder core and/or the chewing gum coat or the film coating may be present in a total amount from about 1.0 to about 8.0 mg (calculated per piece of gum), such as about 1.0 to about 6.0 such as about 2.0 to about 6.0 or about 2.0 to about 5.0 mg, or about 3.0 to about 6.0 or about 3.0 to about 5.0, such as 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0 7.25, 7.5 or 8.0 mg.

The invention also relates to a nicotine chewing gum wherein the powder core comprises nicotine bitartrate and/or nicotine ditartrate dihydrate in an amount of from about 0.5 mg to about 5.0 mg per piece of gum (calculated as the free base), and the gum coat comprises nicotine polacrilex in an amount of from about 0.25 mg to about 6.0 mg (calculated as the free base).

Amounts of nicotine bitartrate and/or nicotine ditartrate dihydrate in the powder core and amount of nicotine polacrilex in the gum coat may be calculated as equivalent of nicotine free base, and expressed in mg per piece of gum.

The gum base in the gum coat according to the invention is about 15-80% by weight of the total gum coat, and preferably at least about 40% by weight.

The amount of gum base employed for the most desirable slow release of nicotine is usually in the higher ranges when nicotine is employed per se or when an absorbed form is used. The gum base may be of any conventional nature known in the art. For example it may comprise a gum base of natural or synthetic origin readily available from a commercial source. Natural gum bases include e.g. chicle, jelutong-, lechi de caspi-, soh-, siak-, katiau-, sorwa-, balata-, pendare-, malaya-, and peach gums, natural cautchouc and natural resins such as dammar and mastix. Synthetic gum bases are a mixture of:
elastomers (polymers, masticating substances),
plasticizer (resin, elastomers, solvent, hydrophobic resin),
filler (texturizer, water-insoluble adjuvant), softener (fat),
emulsifier,
wax,
antioxidant, and
anti-tacking agents (vinyl polymer, hydrophilic resin). Other examples of gum bases are gums including agar, alginate, arabic gum, carob gum, carrageenan, ghatti gum, guar gum, karaya gum, pectin, tragacanth gum, locust beam gum, gellan gum and xanthan gum.

The gum base and the nicotine polacrilex may be present in two distinct layers, said two layers constituting the gum coat. The nicotine polacrilex containing layer may be the inner part of the gum coat, i.e. the nicotine polacrilex containing layer may be in contact with the powder core. Alternatively, the gum base containing layer may be inner part of the gum coat, i.e. the gum base containing layer may be in contact with the powder core. The gum coat may comprise a mixture of the nicotine polacrilex and the gum base; both of which may be in contact with the powder core.

The gum base may not be present in the powder core.

The polyol based filling agents and/or polyol based hard coating may be based on erythritol, maltitol, xylitol, lactitol, isomalt, mannitol, arabitol and sorbitol or mixtures thereof. The polyol based filling agents and/or polyol based hard coating may preferably be xylitol.

The polyol based filling agents may be based on erythritol, maltitol, xylitol, lactitol, isomalt, mannitol, arabitol and sorbitol or mixtures thereof. The polyol based filling agents may preferably be xylitol.

The polyol based hard coating may be based on erythritol, maltitol, xylitol, lactitol, isomalt, mannitol, arabitol and sorbitol or mixtures thereof. The polyol based hard coating may preferably be xylitol.

In one embodiment of the nicotine chewing gum with the powder core, the powder core may comprise Nicotine hydrogen tartrate (NHT), sodium carbonate, sodium bicarbonate, xylitol and neotame.

The nicotine chewing gum could be coated with an outer polyol-based hard coating and may further include one or more polymer based film coatings.

The individually film coatings may have a thickness of from about 20 μm to about 200 μm, such as 20 μm to about 100 μm, such as 20 μm to 60 μm, such as 30 μm.

The film forming polymers may be chosen among hydroxy propyl methyl cellulose (HPMC), methyl hydroxy ethyl cellulose (MHEC), hydroxy propyl cellulose (HPC), hydroxyethyl cellulose (HEC), methacrylic acid copolymer-type C, sodium carboxy methyl cellulose, hydroxypropyl methylcellulose phthalate, (HPMCP), ethyl hydroxyl ethyl cellulose (EHEC), and other film forming polymers such as, polydextrose, polyethylene glycols, acrylate polymers, polyvinyl alcohol-polyethylene glycol graft copolymers, complex of polyvinylpyrrolidone (PVP), such as povidone, polyvinyl alcohol (PVOH or PVA), microcrystalline cellulose, carrageenan, pregelatinized starch, polyethylene glycol, and combinations thereof.

In one embodiment, the film-forming polymers are selected among hydroxy propyl methyl cellulose (HPMC), methyl hydroxy ethyl cellulose (MHEC), hydroxy propyl cellulose (HPC), hydroxyethyl cellulose (HEC), ethyl hydroxyl ethyl cellulose (EHEC) and polyvinyl alcohol (PVOH or PVA).

If two polymer based film coatings are applied, the same as well as different polymers and mixtures thereof could be used in the separate coatings. In one embodiment, both film coatings (also referred to as film layers) may be based on hydroxy propyl methyl cellulose (HPMC). In another embodiment, one film layer is based on hydroxy propyl methyl cellulose (HPMC) and the other film layer is based on another film forming polymer such as polyvinyl alcohol (PVOH or PVA). The film forming polymers may display somewhat different characteristics. For example, PVA is regarded to be more resistant against elevated pH and could thus be suitable to be used for the buffer containing film coating when there is an aim for a high pH.

HPMC provides a nice palatability as well as being able to provide a prolonged boost of flavor and sweetener when applied to a hard-coated nicotine chewing gum.

The polyol-based hard coating outside the chewing gum or the film coating may comprise at least one sugar alcohol or mixtures thereof. The sugar alcohol may be erythritol, maltitol, xylitol, lactitol, isomalt, mannitol, arabitol, isomalt and sorbitol or mixtures thereof. The polyol-based hard coating may have a thickness of from about 350 μm to about 750 μm, such as 500 μm. In one embodiment, the polyol-based hard coating is a mixture of xylitol and maltitol or contains xylitol only.

Examples of flavoring agents/flavors include, fruit and berry flavors such as lime, orange, lemon, black current, blood orange, cranberry, cloudberry, goji berry, raspberry, strawberry, wild strawberry, sea buckthorn, cherry, melon, kiwi, papaya, pineapple, passion fruit, coconut, and other flavors such as honey, herbs, the, anise, water grass, lemon grass, cooling agent, ginger, coffee, eucalyptus, mangostan, peppermint, spearmint, wintergreen, tutti-frutti, cinnamon, cacao/cocoa, vanilla, liquorice, salt, pepper, chili, menthol, aniseeds, or mixtures thereof. The flavoring agents/flavors may be natural extracts as well as synthetic and semisynthetic versions as well as mixtures of flavors. The flavors may be the same or different and can be present in the powder core, gum coat, film coating(s) and/or polyol-based hard coating. Suitable examples of flavors are mint family flavors, fruit and berry flavors.

In addition, the chewing gum may have at least one artificial sweetener present in the powder core and/or the gum coat. The at least one artificial sweetener may be present in the gum coat, powder core, film coating(s) and/or polyol-based hard coating. Examples of artificial sweeteners are saccharin, sodium saccharin, aspartame, acesulfame K, neotame, thaumatin, glycyrrhizin, sucralose, cyclamate, dihydrochalcone, alitame, advantame, miraculin and monellin and mixtures thereof.

Nicotine salts in the chewing gum coat may be present in an amount of about 0.25 to about 6 mg, such as 0.5 to about 1 mg or 0.25, 0.5, 0.75, 1.0, 1.25, 1.5, 1.75, 2.0, 2.25 or 2.5 mg.

In addition, the chewing gum coat may have at least one artificial sweetener. The at least one artificial sweetener may be present in the powder core and/or the chewing gum coat. Examples of artificial sweeteners are saccharin, sodium saccharin, aspartame, acesulfame K, neotame, thaumatin, glycyrrhizin, sucralose, cyclamate, dihydrochalcone, advantame, alitame, miraculin and monellin and mixtures thereof. Neotame acts as a taste masker and sweetener and is suitable to mask the unpleasant taste of nicotine.

Finally, the invention relates to the use of above defined nicotine chewing gums for the treatment of a human beings suffering from cravings from tobacco dependency and/or e-cigarette dependency.

The invention relates to the above defined nicotine chewing gums for use in the treatment of a human beings suffering from cravings from tobacco dependency and/or e-cigarette dependency.

### EXAMPLES

Specific embodiments of the present invention are illustrated by way of the following examples. This invention is not confined to the specific limitations set forth in these examples.

The amounts of the ingredients are expressed in different ways in the examples and not consistent with what will result in a final chewing gum. However, for a person skilled in the art it is obvious how to recalculate and develop a chewing gum according to the disclosure.

### EXAMPLE 1 Powder Mixtures for a Core of a Nicotine Chewing Gum

Table with different amounts of nicotine and different amounts of buffers, polyol based filling agent, and sweeteners.

| | Sam- | Sam- | Sam- | Sam- | Sam- | Sam- | Sam- |
|---|---|---|---|---|---|---|---|
| | ple | ple | ple | ple | ple | ple | ple |
| Ingredients | 1 | 2 | 3 | 4 | 5 | 6 | 7 |
| NHT | 3 | 3 | 3 | 3 | | 6 | 3 |
| NRC | | | | | 3 | | |
| Xylitol | 31 | | 31 | 31 | 31 | 31 | 71 |
| Mannitol | | 31 | | | | | |
| Na₂CO₃ | 4 | 4 | 6 | | 4 | | 4 |
| NaHCO₃ | 2 | 2 | | 8 | 2 | | 2 |
| Neotame | 0.02 | 0.02 | | | | | 0.02 |
| Acesulfame | | | | | 0.25 | | |
| K | | | | | | | |
| Sucralose | | | | | 0.5 | | |

NHT = nicotine hydrogen tartrate
NRC = nicotine resinate complex
All amounts are in mg/gum

All the ingredients are mixed and sieved through a mesh of 710 μm to ensure good distribution of the powder mix without lumps/aggregates.

### EXAMPLE 2 Chewing Gum Base and Production of the New Nicotine Chewing Gum

Nicotine chewing gum according to the invention can be produced by conventional gum manufacturing process:

Chewing gum base was obtained from a commercial chewing gum base producer. Preparation of chewing gum base with nicotine polacrilex was made according to the one or more of the processes/examples disclosed in U.S. Pat. No. 3,877,468.

Preparation of the chewing gum with a powder core was made according to one or more of the processes/examples disclosed in U.S. Pat. No. 5,976,581 using a conventional confectionary machine.

WO2006/124366 discloses the process to formulate and manufacture nicotine gums with polymer based film containing inside a polyol-based hard coating.

### EXAMPLE 3 Evaluation of the Nicotine Chewing Gum with Powder Cores According to EXAMPLE 1

5 subjects evaluated the powder mixtures Sample 1-7 from EXAMPLE 1, wherein the powders were present within chewing gums prepared according to EXAMPLE 2. Time to craving relief was measured and found to be between 10 to 30 seconds depending on how the subject chewed on the nicotine chewing gum.

In addition the subjects had to score the different products (low score being 1 and high score 5) for the mouth feeling and if they could feel the powder. High score 5 meaning nice palatability and no powder feeling.

All subjects gave the score 5 for Samples 1-7. There was no difference if xylitol or mannitol, different buffers or sweetener were used. To get statistical significance this needs to be repeated with a larger group of subjects but gives an indication.

1. A nicotine chewing gum comprising:
i. a powder core comprising at least one of nicotine bitartrate and/or nicotine ditartrate dihydrate, a sweetener, a buffer and a polyol-based filling agent; and
ii. a gum coat comprising at least a gum base and nicotine polacrilex,
wherein the nicotine bitartrate and/or nicotine ditartrate dihydrate from the powder core has an immediate release and the nicotine polacrilex from the gum coat has an extended release.

2. The nicotine chewing gum according to claim 1, wherein the powder core is present in an amount of up to 80 mg and the gum coat is present in an amount of about 950 mg.

3. The nicotine chewing gum according to claim 1, wherein the powder core is present in an amount of about 20 mg to about 60 mg.

4. The nicotine chewing gum according to claim 1, further comprising at least one polyol-based hard coating that coats an exterior of the gum coat.

5. The nicotine chewing gum according to claim 1, wherein the sweetener is selected from the group consisting of a saccharin, sodium saccharin, aspartame, acesulfame K, neotame, thaumatin, glycyrrhizin, sucralose, cyclamate, dihydrochalcone, advantame, alitame, miraculin and monellin and mixtures thereof.

6. The nicotine chewing gum according to claim 1, wherein the buffer is selected from the group consisting of sodium carbonate, sodium bicarbonate, potassium carbonate, potassium bicarbonate, trometamol base (Tris base) or the corresponding conjugated acid of trometamol such as Trometamol hydrochloride (Tris HCl), trisodium phosphate, disodium hydrogenphosphate, sodium dihydrogen phosphate, tripotassium phosphate, dipotassium hydrogenphosphate, potassium dihydrogen phosphate, and mixtures thereof.

7. The nicotine chewing gum according to claim 6, wherein the buffer is sodium carbonate and/or sodium bicarbonate.

8. The nicotine chewing gum according to claim 4, wherein the polyol-based filling agent and/or the polyol-based hard coating are selected from the group consisting of xylitol, erythritol, lactitol, mannitol, maltitol, isomalt, arabitol and sorbitol or mixtures thereof.

9. The nicotine chewing gum according to claim 1, wherein the powder core comprises nicotine bitartrate and/or nicotine ditartrate dihydrate in an amount of from about 0.5 mg to about 5.0 mg per piece of gum (calculated as the free base), and the gum coat comprises nicotine polacrilex in an amount of from about 0.25 mg to about 6.0 mg (calculated as the free base).

10. Use of the nicotine chewing gum according to claim 1 for the treatment of a human being suffering from cravings from tobacco and/or e-cigarette dependency.

11. The nicotine chewing gum according to claim 1, wherein
the sweetener is selected from the group consisting of a saccharin, sodium saccharin, aspartame, acesulfame K, neotame, thaumatin, glycyrrhizin, sucralose, cyclamate, dihydrochalcone, advantame, alitame, miraculin and monellin and mixtures thereof;
the buffer is selected from the group consisting of sodium carbonate, sodium bicarbonate, potassium carbonate, potassium bicarbonate, trometamol base (Tris base) or the corresponding conjugated acid of trometamol such as Trometamol hydrochloride (Tris HCl), trisodium phosphate, disodium hydrogenphosphate, sodium dihydrogen phosphate, tripotassium phosphate, dipotassium hydrogenphosphate, potassium dihydrogen phosphate, and mixtures thereof; and
the polyol-based filling agent is selected from the group consisting of xylitol, erythritol, lactitol, mannitol, maltitol, isomalt, arabitol and sorbitol or mixtures thereof.
